(19) United States
(12) Patent Application Publication
(10) Pub. No.: US 2025/0121100 A1
(43) Pub. Date: Apr. 17, 2025

(54) COMPOSITION AND METHOD FOR DIAGNOSTIC IMAGING

(71) Applicant: Suntec Medical, Inc., Walnut, CA (US)

(72) Inventors: Yuan-Chung Tsai, Taipei (TW); Chun-Ting Cheng, Taipei (TW); Pauline Ying Lau, Walnut, CA (US)

(21) Appl. No.: 18/988,113

(22) Filed: Dec. 19, 2024

Related U.S. Application Data
(63) Continuation of application No. PCT/US2023/068816, filed on Jun. 21, 2023.
(60) Provisional application No. 63/355,247, filed on Jun. 24, 2022.

(51) Int. Cl. A61K 49/18; A61K 49/10; A61K 51/12

(52) U.S. Cl. CPC A61K 49/1809; A61K 49/103; A61K 49/106; A61K 51/1227; A61K 2123/00

(57) ABSTRACT

The present invention provides compositions and methods for diagnostic imaging. The composition comprises micelles having an outer shell comprising one or more polymer-flavonoid conjugates, optionally an inner shell comprising one or more flavonoid oligomer, and a diagnostic imaging agent encapsulated within the shells. The present invention also provides methods for performing diagnostic imaging using the compositions.

### CROSS-REFERENCE TO RELATED APPLICATION(S)

This application is a continuation of PCT/US2023/068816, filed Jun. 21, 2023; which claims the benefit of U.S. Provisional Application No. 63/355,247, filed Jun. 24, 2022. The contents of the above-identified applications are incorporated herein by reference in their entireties.

### FIELD OF THE INVENTION

The present invention provides compositions for diagnostic imaging. The composition comprises micelles having an outer shell formed by one or more hydrophilic polymer-flavonoid conjugates, optionally having an inner shell formed by one or more flavonoid oligomers, and having a contrast agent encapsulated within the shell. The invention also provides methods for performing diagnostic imaging using the compositions.

### BACKGROUND OF THE INVENTION

Imaging modalities including computed tomography (CT), magnetic resonance imaging (MRI), positron emission tomography (PET), single-photon emission computed tomography (SPECT), ultrasound (US), and photoacoustic (PA) are widely used for disease diagnosis, treatment efficacy assessments and disease progression monitoring.

### Computed Tomography (CT)

CT is a potent diagnostic imaging modality that is low expensive, deep tissue permeation, great spatial, and high resolution. CT scan usages are:
Brain or head CT scans: Check for stroke, bleeds, masses, and other abnormalities and examine the skull
Chest CT scans: Provide further insight into abnormalities after a standard chest x-ray
Neck CT scans: Look for enlarged glands or lymph nodes and study lumps.
Spine CT scans: Detect spine problems like spinal canal narrowing, a herniated disc or fractures
Sinus CT scans: Detect and diagnose obstructions or sinus disease
Pelvic or abnormal CT scans: Check organs in this area and diagnose unexplained pain in the abdomen

However, the low signal-to-noise ratio reduces the ability of CT to distinguish between neighboring tissues. For this reason, several heavy atoms such as iodine, tungsten, and barium are suitable as contrast media in CT images due to their great x-ray attenuation coefficient. Although these disadvantages can be compensated by applying contrast media, the chemical stability, solubility, denseness, and toxicity are still a tough challenge to clinical applications. CT contrast agents include but not limited to iohexol, iodixanol, iopamidol, iopromide, ioversol, ioxilan, cholografin meglumine, conray, conray 30, conray 43, cysto-conray II, cysto-conray, cystografin dilute, cystografin, gastrografin, renografin-76, ethiodol, hexabrix, or isovue, diatrizoate sodium, meglumin, ioxaglate, ioxaglate, ioxaglate sodium, iothalamate, iron oxide, iothalamate sodium, Au@BSA, gold nanoparticles, Bi-DTPA, N1177, dextran-coated cerium oxide nanoparticles, Bi-NU-901, PVB-Bi2S3, Er3+-doped Yb203, FePt nanoparticles, AuNPs-PEG, metrizoate, iobitridol, barium or carbon dioxide.

### Magnetic Resonance Imaging (MRI)

The excellent features of MR imaging, as molecular imaging, include comparably high temporal and spatial resolution, excellent tissue contrast and tissue penetration, no ionizing radiation, non-invasiveness for serial studies, and simultaneous acquisition of anatomical structure and physiological function. MRI usages include Spinal cord and brain anomalies, Cysts, tumors other bodily irregularities, Joint abnormalities and injuries, Breast tissue to screen for cancer, A woman's pelvic area to identify issues like fibroids and endometriosis, Suspected uterine anomalies, Abdominal or liver diseases.

However, a limitation of MR imaging is low sensitivity which entails the introduction of an imaging contrast agent and the development of powerful signal amplification strategies. MRI contrast agents include but not limited to gadopentetate, gadoterate, gadobutrol, gadoteridol, gadobenate, gadoxetate, gadoversetamide, gadodiamide, gadofosveset, gadopentetic acid dimeglumine, gadoxentate, gadocoletic acid, gadomelitol, gadomer 17, gadoxetic acid, gadoterate meglumine, gadoxetate disodium, gadofosveset trisodium, mangafodipir, gadobenate dimeglumine, ferumoxsil, ferumoxides, iron oxide, EP-3533, ManlCS1, USPIO-g-sLex, MS-325, PVP-IO (polyvinylpyrrolidone (PVP)-coated iron oxide), ProCA (protein-based MRI contrast agents), SPION, SPION-AN-FA, or Fe3O4.

### Positron Emission Tomography (PET) and Single-Photon Emission Computed Tomography (SPECT)

PET has high sensitivity, limitless depth of penetration, and quantitative capabilities. It becomes a powerful method for cancer diagnosis and functional imaging of other abnormalities. PET is more sensitive in diagnosing cancer tissues when the cancer tissue is small in size not detectable by either CT or MRI, especially when the cancerous tissues are still embedded in the organ and not budding out to the surface of an organ to be detected by MRI or CT. It is often used to follow up at the early stages of cancer cell regrowth after treatments.

The most useful imaging contrast agent for cancer among a variety of radiopharmaceuticals for molecular and metabolic imaging with PET is the fluorodeoxyglucose. Following intravenous injection, FDG (2-deoxy-2-[18F] fluoroglucose), similarly to normal glucose, is taken up by cancer cells. The subsequent conversion of FDG to FDG-6-monophosphate by the intracellular enzyme hexokinase leads to the trapping of the metabolite within the cancer cells. On the other hand, SPECT as an excellent nuclear imaging technique, which is based on the detection of gamma-ray photons, is utilized for imaging due to its fast detection time, specificity, and affordability as compared to PET. However, SPECT is generally less sensitive than PET and also has lower spatial resolution compared to PET. In comparison to CT, the amount of radiation exposure from PET or SPECT is significantly increased due to the radionuclide continuously releasing high-energy gamma-rays or positrons. Therefore, reducing the radionuclide dose usage while maintaining the contrast ability is a critical issue for PET/SPECT contrast agent development.

PET agents include but not limited to 89Zr, Rubidium chloride Rb-82, neuraceq, vizamyl, florbetapir F-18, choline C-11, amyvid, Ga-68 dotatate, axumin, flutemetamol F-18, cardiogen-82, florbetaben F-18, fluciclovine F-18, Ruby-Fill, cerianna, netspot, Ga-68 dotatoc, tauvid, Ga-68 psma-11, detectnet, fluoroestradiol F-18, Cu-64 dotatate, flortaucipir F-18, piflufolastat F-18, pylarify, illuccix, or locametz, C-11, Ga-68, C11-PiB, Cu-64 ATSM, LMI1195, F-18 TFB, F-18 FSPG, F18-FDS, Ga-68-apo-transferrin, F-18 AV-133, F-18 AV-45, F-18-T808, F-18-T807, or F-18-GE-180.

SPECT can be used in oncology, neuroimaging, cardiology, infectious diseases, bio-distribution studies musculo-skeletal imaging. SPECT agents include but not limited to gallium (III), Tc99m, I-131, Tc-99m MDP, Tc-99m MAA, Tc-99m PYP, Tc-99m sulfur colloid, I-131 metaiodobenzyguandine (MIBG), T1-201, Ga-67, I-123, Tc-99m 04, TcO4-, Tc-99m phytate, Tc-99m DISIDA, Tc-99m DTPA, Tc-99m MAG3, Tc-99m DMSA, Tc-99m HMPAO, Tc-99m ECD, Tc-99m MIBI, Tc-99m sestamibi, T1-201, 1-131 OIH, I-131 6b-iodomethyl-19-norcholesterol (NP59), Ga-67, Xe-133, Kr-81m, In-111, or I-123 IMP, Lu-177, I-123 iodoamphetamine, Cu-64, As-72, Zr-89, I-124, C-11, Ga-68, F-18, Tc-99m CN5DG, Ro 16-0154, In111-DOTA-5D3, Tc99m-HYNIC-TMTP1 or QT-DTC-bisbiotin.

### Ultrasound Imaging

Ultrasound imaging is a non-invasive imaging modality with high soft-tissue contrast and without exposing the patient to radiation. It has been used to classify benign, solid lesions with a negative predictive value of 99.5% and can be applied for both imaging and therapeutic purposes. In this imaging modality, different types of bubbles are used as imaging contrast agents with sizes of nano- to micro-meters. Unfortunately, the properties such as size distribution and stability of these bubbles are significantly affected by physiological conditions.

Ultrasound agents include but not limited to Albunex, Bisphere, Luminity, Echogen, Echovist, Filmix, Imavist, Levovist, Myomap, Optison, Quantison, Sonavist, Sonazoid, SonoGen, SonoVue, Lumason, PB127.

Ultrasound scan can be used for: heart, joints, uterus, blood vessels, muscles, bladder, kidneys and more.

### Photoacoustic (PA) Imaging

PA imaging is an emerging technique that has immense potential for augmenting ultrasound with rich optical contrast and can serve as a portable and relatively low-cost standalone modality for regional imaging. The core strengths of PA image are its potential for high spatial/temporal resolution, clinically relevant imaging depth, ability to image both endogenous and exogenous chromophores, and the absence of ionizing radiation. Common endogenous chromophores include water (both free and bound), oxyhemoglobin (HbO2), deoxyhemoglobin (Hb) melanin, and lipids. Exogenous agents are mostly small molecule dyes such as Indocyanine green (ICG), Methylene Blue Dye (MBD), nanoparticles, or even reporter gene agents. Unlike current microbubble-based ultrasound contrast agents, PAI can image small molecules that can readily extravasate, target cell membrane molecules, or even enter cells of interest to target intracellular molecules, and thus provides the clinician with potentially valuable molecular data.

PA agents include but not limited to ICG, CuS, WS2, MoS2, Ag2S, Co9Se8, ZnS, Nb2C, Bi2S3, carbon dots, indocyanine green, methylene blue, IR800-dye, Evans blue.

PA can be used for brain lesion detection, hemodynamics monitoring, breast cancer diagnosis and more.

The above-mentioned imaging modalities suffer from insufficient contrasting ability to differentiate between normal tissue and disease lesions and require contrast agents to enhance its imaging differences. To achieve the required differences in imaging, patients receive multiples of grams of these contrast agents which cause serious adverse effects.

### Flavonoids

Flavonoids have the general structure of a 15-carbon skeleton, which consists of two phenyl rings (A and B) and a heterocyclic ring (C, the ring containing the embedded oxygen).

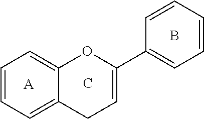

This carbon structure can be abbreviated C6-C3-C6. According to the IUPAC nomenclature, flavonoids can be classified into:
flavonoids or bioflavonoids
isoflavonoids, derived from 3-phenylchromen-4-one (3-phenyl-1,4-benzopyrone) structure
neoflavonoids, derived from 4-phenylcoumarine (4-phenyl-1,2-benzopyrone) structure

### BRIEF DESCRIPTION OF THE DRAWINGS

FIG. 1 illustrates one embodiment of a MINC (Multi-pathway Immune-modulating Nanocomplex Combination therapy)-agent, which is a micelle having a polymer-flavonoid conjugate, for example, a PEG-EGCG conjugate, in a shell and having an agent encapsulated.

FIG. 2 illustrates another embodiment of a MINC-agent, which is a micelle comprises a polymer-flavonoid conjugate, e.g., a PEG-EGCG conjugate in an outer shell and a flavonoid oligomer, for example, oligomeric EGCG (OEGCG), in an inner shell, and having an agent encapsulated.

FIG. 3 shows a successful formulation of MINC-iohexol, MINC-iopamidol, MINC-diatrizoate and MINC-metrizoate.

FIG. 4 shows a successful formulation of MINC-gadopentetate, MINC-gadodiamide, MINC-gadoxetate and MINC-gadoterate.

FIG. 5 shows that MINC-gadopentetate enhanced tumor contrast signal more than gadopentetate dimeglumide in tumor-bearing mouse model.

### DETAILED DESCRIPTION OF THE INVENTION

### Definitions

The term “epigallocatechin gallate” refers to an ester of epigallocatechin and gallic acid, and is used interchangeably with “epigallocatechin-3-gallate” or EGCG.

The term “oligomeric EGCG” (OEGCG) refers to 2-50, 3-20 monomers of EGCG that are covalently linked. OEGCG preferably contains 4 to 12 monomers of EGCG.

The term “polyethylene glycol-epigallocatechin gallate conjugate” or “PEG-EGCG refers to polyethylene glycol (PEG) conjugated to one or two molecules of EGCG. The term “PEG-EGCG” refer to both PEG-mEGCG conjugate (monomeric EGCG) and PEG-dEGCG (dimeric EGCG) conjugate.

The present invention provides a diagnostic imaging composition for enhancing the signal of a diagnostic imaging technology for the aids of monitoring, prognosing and diagnosing a disease so as to make a disease treatment plan. The composition comprises micelles having an outer shell formed by one or more hydrophilic polymer-flavonoid conjugates, optionally having an inner shell formed by one or more flavonoid oligomers, and having an imaging agent encapsulated within the shell.

### Flavonoids

Flavonoids suitable for the present invention have the general structure of Formula I:

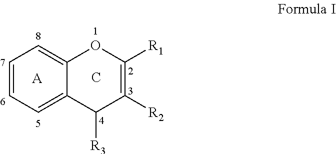

wherein:
R₁ is H, or phenyl;
R₂ is H, OH, Gallate, or phenyl; wherein the phenyl is optionally substituted by one or more (e.g., 2-3) hydroxyl;
R₃ is H, OH, or ═O (oxo); or
R₁ and R₂ together form a close-looped ring structure; or
R₂ and R₃ together form close-looped ring structure.

The 2, 3, 4, 5, 6, 7, or 8 position of Formula I, can be linked to a group containing hydrocarbon, halogen, oxygen, nitrogen, sulfur, phosphorus, boron or metals.

Examples of flavonoids of Formula I include:

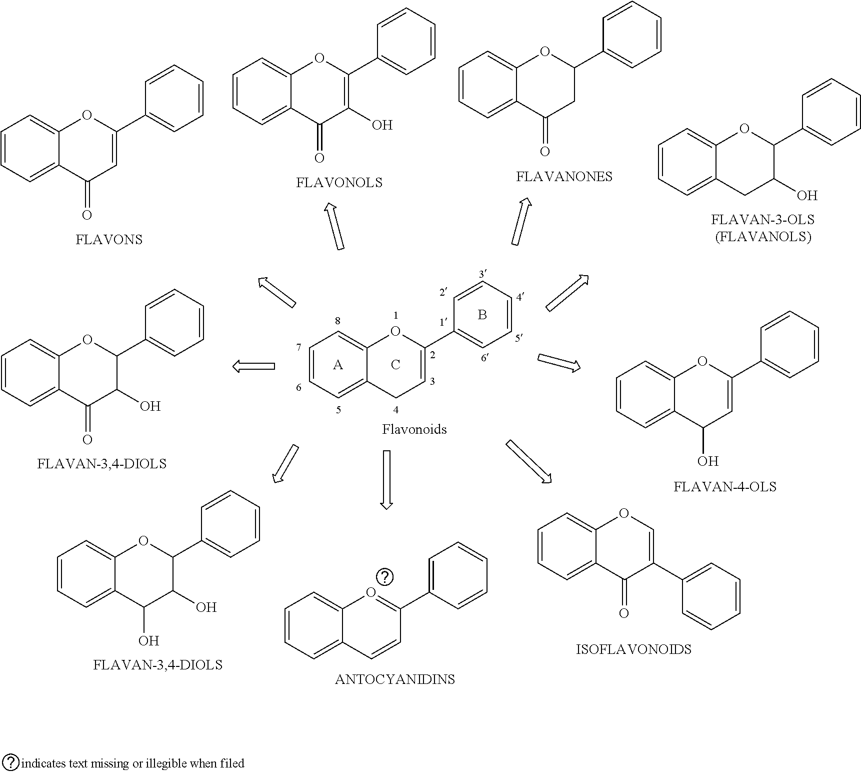

Preferred flavonoid compounds of Formula I include:

### EGCG (CAS #989-51-5), EC (CAS #490-46-0), EGC (CAS #970-74-1) or ECG (CAS #1257-08-5)

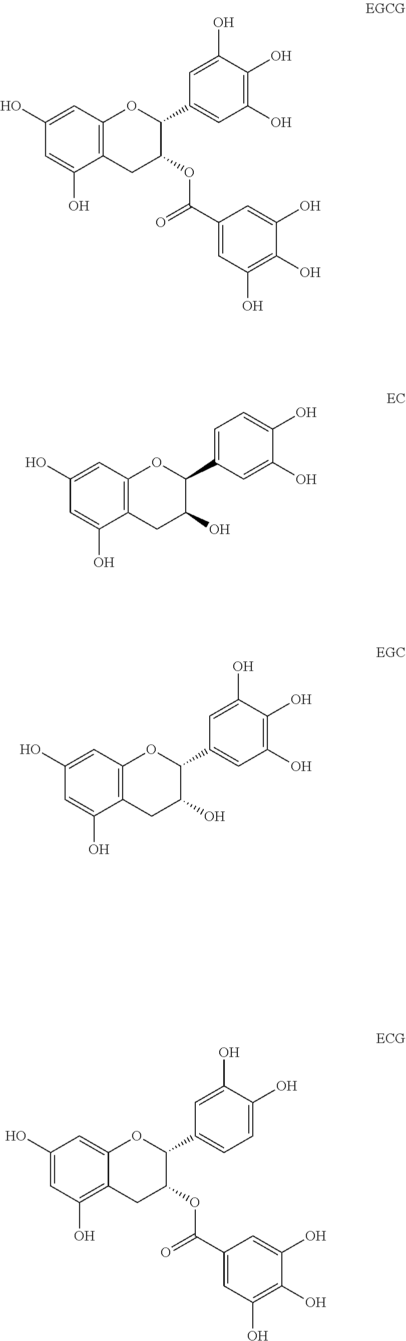 [SMILES: O=C(O[C@@H]1Cc2c(O)cc(O)cc2O[C@@H]1c1cc(O)c(O)c(O)c1)c1cc(O)c(O)c(O)c1 ; Oc1cc(O)c2c(c1)O[C@@H](c1ccc(O)c(O)c1)[C@@H](O)C2 ; Oc1cc(O)c2c(c1)O[C@H](c1cc(O)c(O)c(O)c1)[C@H](O)C2 ; O=C(O[C@@H]1Cc2c(O)cc(O)cc2O[C@@H]1c1ccc(O)c(O)c1)c1cc(O)c(O)c(O)c1]

### A Hydrophilic Polymer-Flavonoid Conjugate

A hydrophilic polymer-flavonoid conjugate, as used herein throughout the application, refers to a conjugate of a hydrophilic polymer and the flavonoid compound of Formula I.

A hydrophilic polymer refers to a polymer that is soluble in polar solvents and can form hydrogen bonds. Hydrophilic polymers suitable for the present polymer-flavonoid conjugates include, but not limited to: poly(ethylene glycol), aldehyde-derivatized hyaluronic acid, hyaluronic acid, dextran, diethylacetal conjugate (e.g. diethylacetal PEG), D-alpha-tocopheryl polyethylene glycol succinate, aldehyde-derivatized hyaluronic acid-tyramine, hyaluronic acid-aminoacetylaldehyde diethylacetal conjugate-tyramine, cyclotriphosphazene core phenoxymethyl(methylhydrazono)dendrimer or thiophosphoryl core phenoxymethyl(methylhydrazono)dendrimer. acrylamides, oxazolines, imines, acrylic acids, methacrylates, diols, oxiranes, alcohols, amines, anhydrides, esters, lactones, terephthalate, amides and ethers polyacrylamide, poloxamers, poly(N-isopropylacrylamide), poly(oxazoline), polyethylenimine, poly(acrylic acid), polymethacrylate, poly(ethylene glycol), poly(ethylene oxide), poly(vinylalcohol), poly(vinylpyrrolidinone), polyethers, poly(allylamine), polyanhydrides, poly(β-amino ester), poly(butylene succinate), polycaprolactone, polycarbonate, polydioxanone, poly(glycerol), polyglycolic acid, poly(3-hydroxypropionic acid), poly(2-hydroxyethyl methacrylate), poly(N-(2 hydroxypropyl)methacrylamide), polylactic acid, poly(lactic-co-glycolic acid), poly(ortho esters), poly(2 oxazoline), poly(sebacic acid), poly(terephthalate-co-phosphate), povidone and copolymers.

Preferred hydrophilic polymers include poly(ethylene glycol), hyaluronic acid, dextran, polyethylenimine, poloxamers, povidone, D-alpha-tocopheryl and polyethylene glycol succinate. The molecular weight of the hydrophilic polymer in the polymer-flavonoid conjugate is in general 1K-100K, preferably 2K-40K, 2K-50K, 2K-80K, 3K-80K, or 5K-40K Daltons. In one embodiment, the polymer contains an aldehyde group which is conjugated to the 5, 6, 7, or 8 position (preferably 6 or 8 position) of the A ring of the flavonoid compound.

In one embodiment, the polymer contains a thiol group which is conjugated to R₁ or R₂ of the B-ring of a flavonoid (when R₁ or R₂ is —OH).

A poly(ethylene glycol) (PEG)-flavonoid conjugate, as used herein throughout the application, refers to a conjugate of PEG and the flavonoid compound of Formula I. The molecular weight of PEG in the PEG-flavonoid conjugate is in general 1K-100K, preferably 3K-80K, and more preferably 5K-40K.

In one embodiment, PEG contains an aldehyde group which is conjugated to the 5, 6, 7, or 8 position (preferably 6 or 8 position) of the A ring of the flavonoid compound. In another embodiment, PEG contains a thiol group which is conjugated to R₁ or R₂ of the B-ring of a flavonoid (when R₁ or R₂ is —OH).

In one embodiment, the PEG-flavonoid conjugate is PEG-EGCG, which is PEG conjugated to one or two molecules of epigallocatechin gallate (EGCG). PEG-EGCG, for example, can be prepared by conjugating aldehyde-terminated PEG to EGCG by attachment of the PEG via reaction of the free aldehyde group with the 5, 6, 7, or 8 position (preferably 6 or 8 position) of Formula I. See WO2006/124000 and WO2009/054813. PEG-EGCG can also be prepared by conjugating thio-terminated PEG to EGCG by attachment of the PEG via reaction of the free thio group with the R₁ or R₂ of Formula I, wherein, R₁ or R₂ is a phenyl group. See WO2015/171079.

### Flavonoid Oligomers

A flavonoid oligomer is a conjugate of one flavonoid with one or more flavonoids. The flavonoid oligomer can contain the same flavonoid (a homo oligomer) or different flavonoids (a hetero oligomer). Flavonoid oligomers useful for the present invention in general have 2-20, preferably 4-12 flavonoids of one or mixed types.

In some embodiment, a flavonoid oligomer is oligomeric EGC (OEGCG), oligomer EC (OEC), oligomer EGC (OEGC) or oligomer ECG (OECG). OEGCG refers to 3-20 monomers of EGCG that are covalently linked. OEGCG, for example, can be synthesized at 5, 6, 7, or 8 position (preferably 6 or 8 position) of the A ring according to WO2006/124000.

Because A-ring is present in all of the flavonoids according to Formula 1, other oligomeric flavonoids can be made similarly according to WO2006/124000. For example, OEC, OEGC, and OECG can also be made according to WO2006/124000.

### MINC-Agent

MINC (Multi-pathway Immune-modulating Nanocomplex Combination therapy) is a platform technology, utilizing the bioactivity of a hydrophilic polymer-flavonoid conjugates and/or flavonoid oligomers that form micelles in a solution. The present application using MINC platform to encapsulate additional imaging agents to form a nanoparticle composition for diagnostic imaging.

MINC-agent is a micelle with a shell formed by one or more hydrophilic polymer-flavonoid conjugates, optionally with one or more flavonoid oligomers, and has an imaging agent encapsulated within the shell. The imaging agent, as used herein, reference to a molecule that can enhance the contrasting quality of an imaging technology by the MINC technology.

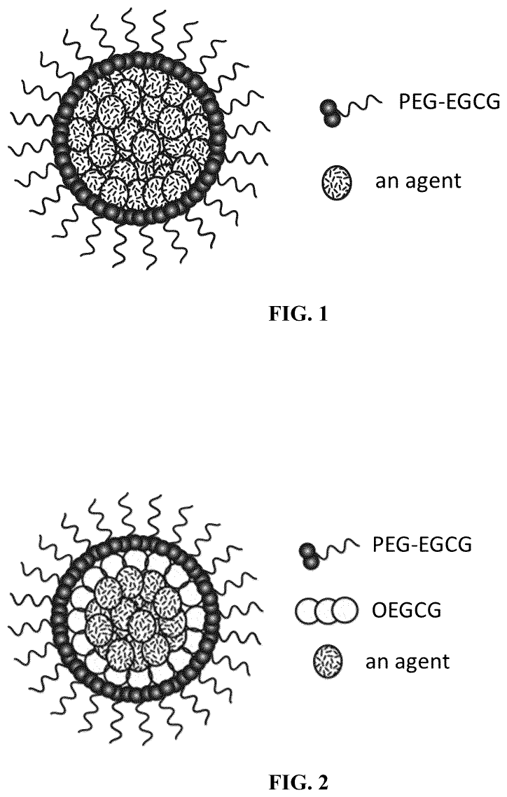

In one embodiment, MINC-agent is a micelle comprises hydrophilic polymer-flavonoid conjugates, e,g., PEG-EGCG conjugates, in a shell and with an imaging agent encapsulated (see FIG. 1).

In another embodiment, MINC-agent is a micelle comprises hydrophilic polymer-flavonoid conjugates, e,g., PEG-EGCG conjugate in an outer core and flavonoid oligomers, e.g., oligomeric EGCG (OEGCG), in an inner core, with an imaging agent encapsulated (see FIG. 2).

In the present application, MINC-agent can be used for computed tomography (CT), magnetic resonance imaging (MRI), positron emission tomography (PET), single-photon emission computed tomography (SPECT), ultrasound, and photoacoustic (PA) imaging with similar injection procedure as traditional contrast agents. Compared to a traditional contrast agent, MINC-agent has the advantages: (1) prolonged residence time in tissues, (2) more accumulation in the targeted sites via enhanced permeability and retention (EPR) effect and (3) less accumulation in off-target site with reduced toxicity. Taken together, MINC-agent has higher signal intensity and is safer than traditional imaging agents.

Imaging agents in MINC-agents for CT include but not limited to iohexol, iodixanol, diatrizoate, metrizoate, iopamidol, iopromide, ioversol, ioxilan, cholografin meglumine, conray, conray 30, conray 43, cysto-conray II, cysto-conray, cystografin dilute, cystografin, gastrografin, renografin-76, ethiodol, hexabrix, or isovue, meglumin, ioxaglate, ioxaglate, ioxaglate sodium, iothalamate, iron oxide, iothalamate sodium, Bi-DTPA, N1177, Bi-NU-901, PVB-Bi2S3, Er3+-doped Yb203, iobitridol, barium, and carbon dioxide.

Imaging agents in MINC-agents for MRI include but not limited to gadopentetate, gadoterate, gadodiamide, gadoxetate, gadobutrol, gadoteridol, gadobenate, gadoversetamide, gadodiamide, gadofosveset, gadopentetic acid dimeglumine, gadoxentate, gadocoletic acid, gadomer 17, gadoxetic acid, gadoterate meglumine, gadoxetate disodium, gadofosveset trisodium, mangafodipir, gadobenate dimeglumine, ferumoxsil, ferumoxides, iron oxide, EP-3533, ManlCS1, USPIO-g-sLex, MS-325, PVP-IO, ProCA, SPION, SPION-AN-FA, and Fe3O4.

Imaging agents in MINC-agents for PET include but not limited to 89Zr, rubidium chloride Rb-82, neuraceq, vizamyl, florbetapir F-18, choline C-11, amyvid, Ga-68 dotatate, axumin, flutemetamol F-18, cardiogen-82, florbetaben F-18, fluciclovine F-18, Ruby-Fill, cerianna, netspot, Ga-68 dotatoc, tauvid, Ga-68 psma-11, detectnet, fluoroestradiol F-18, Cu-64 dotatate, flortaucipir F-18, piflufolastat F-18, pylarify, illuccix, or locametz, C-11, Ga-68, C11-PiB, Cu-64 ATSM, LMI1195, F-18 TFB, F-18 FSPG, F18-FDS, Ga-68-apo-transferrin, F-18 AV-133, F-18 AV-45, F-18-T808, F-18-T807, and F-18-GE-180.

Imaging agents in MINC-agents for SPECT include but not limited to gallium (III), Tc99m, I-131, Tc-99m MDP, Tc-99m MAA, Tc-99m PYP, Tc-99m sulfur colloid, I-131 metaiodobenzyguandine (MIBG), T1-201, Ga-67, I-123, Tc-99m 04, TcO4-, Tc-99m phytate, Tc-99m DISIDA, Tc-99m DTPA, Tc-99m MAG3, Tc-99m DMSA, Tc-99m HMPAO, Tc-99m ECD, Tc-99m MIBI, Tc-99m sestamibi, Tl-201, 1-131 OIH, I-131 6b-iodomethyl-19-norcholesterol (NP59), Ga-67, Xe-133, Kr-81m, In-111, or I-123 IMP, Lu-177, I-123 iodoamphetamine, Cu-64, As-72, Zr-89, I-124, C-11, Ga-68, F-18, Tc-99m CN5DG, Ro 16-0154, In111-DOTA-5D3, Tc99m-HYNIC-TMTP1 or QT-DTC-bisbiotin.

In one embodiment, the diagnostic imaging composition comprises MINC-agent and one or more pharmaceutically acceptable excipients, which are inactive ingredients suitable to be administered to a subject. Pharmaceutically acceptable excipients can be selected by those skilled in the art using conventional criteria. The pharmaceutically acceptable excipients may contain ingredients that include, but are not limited to, saline and aqueous electrolyte solutions; ionic and nonionic osmotic agents, such as sodium chloride, potassium chloride, glycerol, and dextrose; pH adjusters and buffers, such as salts of hydroxide, phosphate, citrate, acetate, borate, and trolamine; antioxidants, such as salts, acids, and/or bases of bisulfite, sulfite, metabisulfite, thiosulfite, ascorbic acid, acetyl cysteine, cysteine, glutathione, butylated hydroxyanisole, butylated hydroxytoluene, tocopherols, and ascorbyl palmitate; surfactants, such as lecithin and phospholipids, including, but not limited to, phosphatidylcholine, phosphatidylethanolamine and phosphatidyl inositol; poloxamers and poloxamines; polysorbates, such as polysorbate 80, polysorbate 60, and polysorbate 20; polyethers, such as polyethylene glycols and polypropylene glycols; polyvinyls, such as polyvinyl alcohol and polyvinylpyrrolidone (PVP, povidone); cellulose derivatives, such as methylcellulose, hydroxypropyl cellulose, hydroxyethyl cellulose, carboxymethyl cellulose, and hydroxypropyl methylcellulose and their salts; petroleum derivatives, such as mineral oil and white petrolatum; fats, such as lanolin, peanut oil, palm oil, and soybean oil; mono-, di-, and triglycerides; polysaccharides, such as dextrans; and glycosaminoglycans, such as sodium hyaluronate.

### Method of Diagnostic Imaging

### Computed Tomography (CT) Imaging

CT imaging uses x-rays to detect the differential mass density of diseased lesions (e.g. tumor) to the normal tissues in terms of Hounsfield Value (HV), which is calculated from measuring the difference in attenuation of x-rays at disease lesions and different tissues.

A CT contrast agent can enhance CT imaging signal with greater sensitivity than CT alone. A CT contrast agent is often used in tumor detection. Tumor tissue is highly vascular which allows more CT contrast agents to accumulate in tumor than the surrounding tissues and the resulting HV is higher.

In one aspect, the present invention is directed to a method for diagnostic imaging based on CT imaging. The method comprises the steps of: administering to a subject in need thereof an effective amount of micelles as described above, performing CT scan, measuring the difference in attenuating x-rays between lesions and normal tissues, and determining the location and/or size of lesions.

In the present method, the CT scan is performed in whole body, brain, head, chest, neck, spine, sinus, pelvic, or abdomen.

In one embodiment, the lesions are tumors, autoimmune diseases, cardiovascular diseases, central nervous diseases, infection and inflammatory lesions.

In one embodiment, the micelles are administered by intravenous injection, intra-arterial injection, intrathecal injection or oral.

CT contrast agents, for example iohexol, is typically intravenously injected at a concentration of 350 mg/mL with the injection volume of 60-100 mL for the whole body imaging to detect the presence and the size of tumor.

In the present invention, MINC-iohexol is used by IV injection similar to iohexol injection procedure with a similar or less iohexol concentration and injection volume.

Traditional CT imaging timing is restricted to a short 10-15 minutes right after administering the contrast agents, because in a longer time period, the imaging quality is drastically reduced due to fast renal secretion of the contrast agents. MINC-agent imaging period can be extended to 1 or 2 hours or longer after the injection without losing the quality of the imaging signal. This advantage provides much more convenience and accuracy for patient CT scan. MINC-agent can be used with all CT imaging instruments that CT contrast agents apply to enhance contrast signaling and prolonged detection time.

### (Magnetic Resonance Imaging) MRI Imaging

MRI is a medical imaging technique used in radiology to form pictures of the anatomy and the physiological processes of the body. MRI scanners use strong magnetic fields, magnetic field gradients, and radio waves to generate images of the organs in the body. MRI is widely used in hospitals and clinics for medical diagnosis, staging and follow-up of disease. Compared to CT, MRI provides better contrast in images of soft tissues, e.g., in the brain or abdomen.

MRI for imaging anatomical structures or blood flow do not require contrast agents since the varying properties of the tissues or blood provide natural contrasts. However, for more specific types of imaging, exogenous contrast agents may be given intravenously, orally, or intra-articularly. The most commonly used intravenous contrast agents are based on chelates of gadolinium. The MRI contrast agent can improve the visibility of internal body structures in establishing the location and size of the diseased lesions with greater assurance than is possible with MRI alone. An MRI contrast agent is used in measuring organ changes, for example, for tumor detection.

In one aspect, the present invention is directed to a method for diagnostic imaging based on MRI imaging. The method comprises the steps of: administering to a subject in need thereof an effective amount of the micelles as described above in the application, performing MRI, measuring the differences in magnetic resonance signals generated by magnetic fields between lesions and normal tissues to detect lesions; and determining the location and/or size of diseased lesions.

In one embodiment, the lesions are tumors, autoimmune diseases, cardiovascular diseases, central nervous diseases, infection, and inflammatory lesions.

In one embodiment, the imaging organs are brain, breast, spinal cord, bladder, uterus, ovaries, blood vessels, lymph nodes, heart, liver, biliary tract, kidneys, spleen, bowel, pancreas and adrenal glands.

In one embodiment, the micelles are administered intravenously, orally, or intra-articularly.

In the present invention, MINC-gadopentetate dimeglumine is used by IV injection similar to gadopentetate dimeglumine injection procedure and at a similar gadopentetate dimeglumine concentration and injection volume. For example, gadopentetate dimeglumine, is typically IV injected at a concentration of 470 mg/mL and injection volume of 14-18 mL for whole body imaging to detect the presence and size of tumors.

The MRI imaging timing is usually restricted to a short 60-90 minutes right after administering the contrast agents because in a longer time period, the imaging quality is drastically reduced due to fast renal secretion of the contrast agents. MINC-agent imaging period can be extended to 2 or 3 hours or longer after the injection without losing the quality of the imaging signal which provides much more conveniences and accuracy for patient MRI scan. MINC-agent can be used with all MRI imaging instruments that MRI contrast agents apply to enhance contrast signaling and prolonged detection timing.

### Single-Photon Emission Computed Tomography (SPECT) Imaging

SPECT imaging is a nuclear medicine tomographic imaging technique using gamma rays. It is similar to conventional nuclear medicine planar imaging using a gamma camera (that is, scintigraphy), but it is able to provide true 3D information. This information is typically presented as cross-sectional slices through the patient but can be freely reformatted or manipulated as required.

The technique needs delivery of a gamma-emitting radioisotope (a radionuclide) into the patient, normally through injection into the bloodstream. On occasion, the radioisotope is a simple soluble dissolved ion, such as an isotope of gallium(III). Most of the time, a marker radioisotope is attached to a specific ligand to create a radioligand, whose properties bind it to certain types of tissues. This allows the combination of ligand and radiopharmaceutical to be carried and bound to a place of interest in the body, where the ligand concentration is seen by a gamma camera.

A SPECT imaging agent can generate SPECT imaging signal in establishing the location and size of the diseased lesions with better visibility.

In one aspect, the present invention is directed to a method for diagnostic imaging based on SPECT imaging. The method comprises the steps of: administering to a subject in need thereof an effective amount of the micelles as described above in the application, performing SPECT imaging, measuring the differences in signal intensity of gamma rays between lesions and normal tissues, and determining the location and/or size of lesions.

In one embodiment, the diseased lesions include lesions due to tumors, autoimmune diseases, cardiovascular diseases, central nervous diseases, infection, or inflammation.

In one embodiment, the imaging organs are brain, heart, thyroid, bones, lungs, liver, kidneys, parathyroid, gastrointestinal system (stomach, intestines), salivary glands, pancreas, spleen, adrenal glands, prostate, ovaries, testes, blood flow to extremities (arms, legs), lymphatic system, bladder, breasts. In one embodiment, the micelles are administered intravenously.

Compared to traditional imaging agent, MINC-agent can accumulate more in inflammation lesions. Therefore, the signaling intensity and imaging quality is improved more than traditional imaging agent in the diseased lesions. MINC-agent can be used with all SPECT imaging instruments that SPECT imaging agents apply to.

MINC-Tc99m can be used by IV injection similar to the Tc99m injection procedure and at a similar Tc99m concentration and injection volumes. For example, Tc99m, is typically IV injected at a concentration of 20 mCi and injection volume of 1-2 mL for the whole-body imaging to detect the presence and size of tumors, and a concentration of 10 mCi and injection volume of 1-2 mL for the coronary vascular imaging.

### Positron Emission Tomography (PET)

PET is a functional imaging technique that uses radioactive substances known as radiotracers to visualize and measure changes in metabolic processes, and in other physiological activities including blood flow, regional chemical composition, and absorption. Different tracers are used for various imaging purposes, depending on the target process within the body. PET is a common imaging technique, a medical scintillography technique used in nuclear medicine. A radiopharmaceutical—a radioisotope attached to a drug—is injected into the body as a tracer. Gamma rays are emitted and detected by gamma cameras to form a three-dimensional image, in a similar way that an X-ray image is captured.

In one aspect, the present invention is directed to a method for diagnostic imaging based on PET imaging. The method comprises the steps of: administering to a subject in need thereof an effective amount of the micelles as described above in the application, performing positron emission tomography (PET) imaging, measuring three-dimensional image formed by the gamma rays resulted from positron emitted by the imaging agent, and determining the location and/or size of lesions.

In one embodiment, the lesions are tumors, autoimmune diseases, cardiovascular diseases, central nervous diseases, infection, and inflammatory lesions.

In one embodiment, the imaging organs are brain, heart, lung, liver, bone, thyroid, gastrointestinal system, lymphatic system, prostate, ovaries, testes, adrenal gland, soft tissue.

In one embodiment, the micelles are administered intravenously.

MINC-agent can be used with all PET imaging instruments that PET imaging agents apply to. Compared to traditional imaging agents, MINC-agent can accumulate more in inflammation lesions. Therefore, the signaling intensity and imaging quality of MINC-agent is improved over the traditional imaging agents. in the diseased lesions.

PET scanning with the tracer 18F-FDG is widely used in clinical oncology. FDG is a glucose analog that is taken up by glucose-using cells and phosphorylated by hexokinase (whose mitochondrial form is significantly elevated in rapidly growing malignant tumors). Metabolic trapping of the radioactive glucose molecule allows the PET scan to be utilized. MINC-18F-FDG is used by IV injection similar to the 18F-FDG injection procedure and at a similar 18F-FDG concentration and injection volumes. For PET imaging, for example, F18-FDG, is typically IV injected at a concentration of 10 mCi and injection volume of 1-2 mL for the whole-body imaging to detect the presence and size of tumors, and a concentration of 5 mCi and injection volume of 1-2 mL for the coronary vascular imaging.

The following examples further illustrate the present invention. These examples are intended merely to be illustrative of the present invention and are not to be construed as being limiting.

### EXAMPLES

### Active Ingredients

### OEGCG:

OEGCG is oligomerized EGCG. OEGCG is prepared according to WO2006/124000.

### PEG-EGCG:

PEG-EGCG is PEG conjugated with one or two EGCG. PEG-EGCG is prepared according to WO2006/124000, WO2009/054813, or WO2015/171079.

### Minc-Agents:

MINC-agents can be prepared by encapsulated an agent within the micelle formed by PEG-EGCG and OEGCG, according to the method in WO2006/124000 or WO2009/054813. Alternatively, MINC-agents can be prepared by encapsulated an agent within the micelle formed by PEG-EGCG, according to the method in WO2011/112156 or WO2015/171079.

### Example 1. Preparing MINC-Agents for CT Imaging

### Materials

Iohexol, diatrizoate and metrizoate are purchased from Echo Chemical.

Iopamidol is purchased from Wuhan honestchem.

### Method

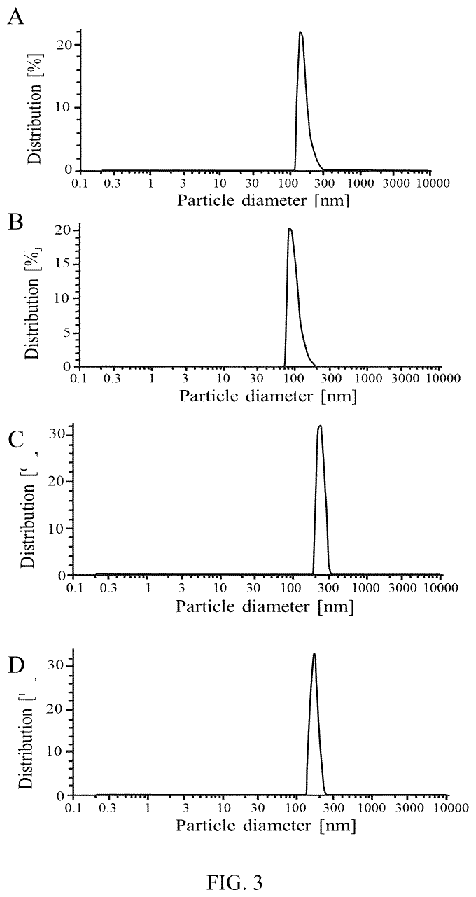

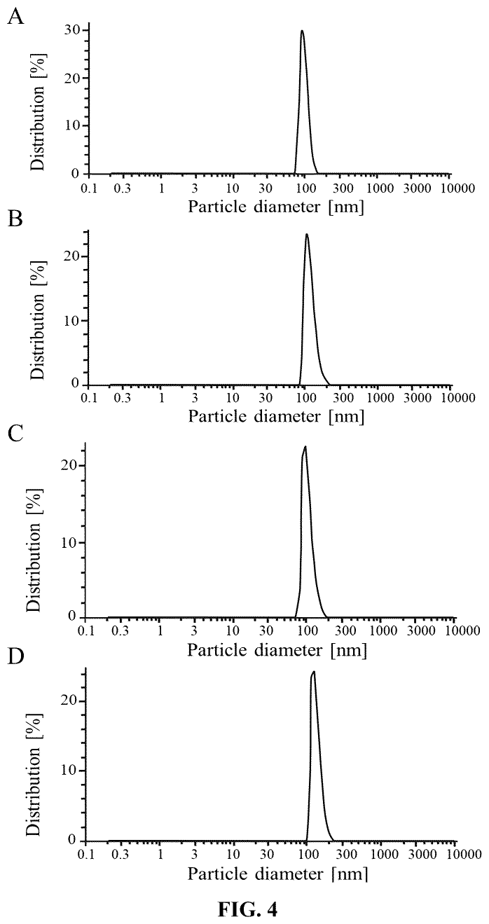

MINC-agents were prepared according to WO2006/124000. In brief, iohexol, iopamidol, diatrizoate and metrizoate were prepared in PBS. Subsequently, flavonoid oligomer OEGCG was added to each contrast agent/PBS, followed by adding polymer-flavonoid PEG-EGCG. After incubating the mixture at room temperature, 10K MWCO centrifugal filter was used to remove the unreacted OEGCG and PEG-EGCG. DLS (Anton Paar Litesizer 500) was used to measure the nanoparticle size and the results are shown in FIG. 3.

### Results

FIG. 3 shows that PEG-EGCG and OEGCG generated MINC-iohexol (A), MINC-iopamidol (B), MINC-diatrizoate (C) and MINC-metrizoate (D) micelles with one single peak of similar particle size around 50 to 300 nm. The results demonstrate that homogenous nanoparticles (micelles) were successfully formed with an expected size, and the unencapsulated agents were not detected by DLS.

These data show that the CT contrast agents, iohexol, diatrizoate, metrizoate and iopamidol were formed by MINC platform.

### Example 2. Preparing MINC-Agents for MRI Imaging

### Materials

Gadopentetate is purchased from Seven Star Pharmaceutical.

Gadodiamide is purchased from Labseeker Inc.

Gadoxetate is purchased from Amadis Chemical.

Gadoterate is purchased from Toronto Research Chemical.

### Method

MINC-agents were prepared according to WO2006/124000. In brief, gadopentetate, gadodiamide, gadoxetate and gadoterate were incubated in PBS. Subsequently, flavonoid oligomer OEGCG was added to the contrast agents, followed by adding polymer-flavonoid PEG-EGCG. After incubating the mixture at room temperature, 10K MWCO centrifugal filter was used to remove the unreacted OEGCG and PEG-EGCG. DLS (Anton Paar Litesizer 500) was used to measure the nanoparticle size and the results are shown in FIG. 4.

### Results

FIG. 4 shows that PEG-EGCG and OEGCG generated MINC-gadopentetate (A), MINC-gadodiamide (B), MINC-gadoxetate (C) and MINC-gadoterate (D) micelles with one single peak of similar particle size around 50 to 300 nm. The results demonstrate that homogenous nanoparticles (micelles) were successfully formed with an expected size, and the unencapsulated agents cannot be detected by DLS.

These data support that the contrast agents, gadopentetate, gadodiamide, gadoxetate and gadoterate, can be formed by MINC platform.

### Example 3. MINC-Gadopentetate Enhanced Contrast Signal than Gadopentetate Dimeglumide for MRI Tumor Imaging

### Materials

MINC-gadopentetate is gadopentetate encapsulated within OEGCG and PEG-EGCG (see Example 2),

LLC1 mouse lung carcinoma cell line was obtained from ATCC, USA.

Male C57BL/6 mice were obtained from Jackson Laboratories, USA.

### Method

This experiment is to confirm the MINC-gadopentetate can be used as a contrast image for tumor detection.

An in vivo xenograft tumor model was used in this experiment. In brief, two male C57BL/6 mice bearing LLC1 mouse lung carcinoma xenografts (6-8 weeks) were divided into two groups (n=1), minimizing their weight differences. The two groups of mice were administered with either gadopentetate or MINC-gadopentetate at a dose of 93.8 mg/kg through intravenous injection. MRI imaging was performed with a BRUKER BIOSPEC 70/30 MRI. Animals were placed prone on the imaging bed with legs secured in an extended position. After the mice were anaesthetized, T1-weighted gradient echo protocol was followed 0.5 h, 2 h and 24 h after the injection. Imaging parameters of the T1-weighted images were TR/TE=8.0/4.2, flip angle=30°, field of view of 50×30 mm, a matrix size of 192×192, and 2 mm of coronal slice thickness, and were TR/TE=8.0/4.5, flip angle=30°, field of view of 45×45 mm, a matrix size of 192×192, and 2 mm of axial slice thickness. For normalized signal intensity relative to the T1-weighted images, the tumor area was selected as a region of interest (ROI). The signal intensity of the ROI was normalized to the intensity of muscle near hip. Three images were taken at each timepoint, and statistics was calculated by student t test. **: p<0.01.

### Result

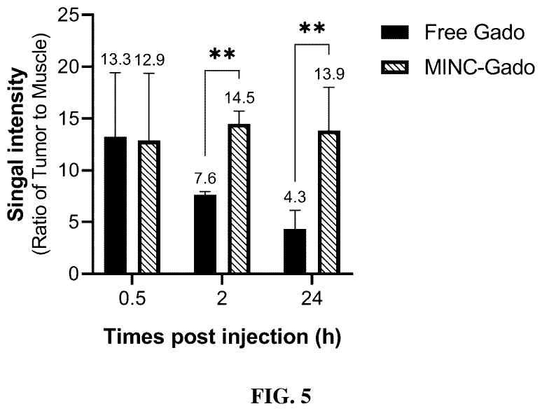

The MRI imaging results demonstrate that MINC-gadopentetate had a higher imaging intensity in tumor than that of free gadopentetate. (FIG. 5).

### Example 4. Comparing Contrasting Signal of MINC-Iohexol and Iohexol for CT Tumor Imaging (Prophetic Example)

### Objective

This experiment is intended to demonstrate the tumor targeting effect of MINC-iohexol. Tumor bearing mice are used to evaluate the signal intensity of MINC-iohexol and iohexol present in tumor. MRI imaging can be used to confirm the efficiency of contrast agent delivered to tumor.

### Materials

MINC-iohexol is iohexol encapsulated within OEGCG and PEG-EGCG and it is prepared according to WO2006/124000.

Iohexol is purchased from Echo Chemical.

MCF-7 human breast cell line is obtained from ATCC, USA

Female athymic nude mice are obtained from Jackson Laboratories, USA.

### Method

To confirm the MINC-iohexol can be used as a contrast image for tumor detection, an in vivo xenograft tumor model is used. In brief, six female athymic nude mice bearing MCF-7 human breast cancer xenografts (6-8 weeks) are divided into two groups (n=3), minimizing their weight differences. The two groups of mice are administered with iohexol and MINC-iohexol at a dose of 0.02 to 100 mg/kg through intravenous injection, respectively. MicroCT imaging performs with a hybrid small-animal scanner (Inveon SPECT/CT; Siemens Medical Solutions USA, Inc.). Animals are placed prone on the imaging bed with legs secured in an extended position. Five minutes after injection, mice undergo high-resolution anatomic CT (360 projections, 80 kVp/500A penetration energy, effective pixel size of 96 m) imaging. The microCT images are reconstructed using the cone-beam algorithm with existing commercial software (Cobra Exxim) and intensity values are converted to Hounsfield units (HU). The quantitative analysis is measured using Inveon Research Workspace (Siemens Medical Solutions USA, Inc.). Briefly, complex irregular volumes of interest (VOIs) are drawn on the microCT images to determine the mean counts in each VOI.

### Example 5. MINC-Gadopentetate is Used as a Contrast Agent for Type 1 Diabetes Detection Using MRI (Prophetic Example)

### Objective

This example is intended to demonstrate that compared to the free gadopentetate, MINC-gadopentetate improves the contract signal of pancreatic islets. MRI imaging is used to confirm contrast signal difference between normal and inflammatory pancreatic islets.

### Materials

MINC-gadopentetate is gadopentetate encapsulated within OEGCG and PEG-EGCG and it is prepared according to WO2006/124000.

Gadopentetate is Obtained from Seven Star Pharmaceutical

Glucosemeter is obtained from Glucometer Elite, Bayer or from other sources.

NOD/Lt, Eα16/NOD, NOD-RAG−/−, BDC2.5/NOD, BDC2.5/B6·H-2g7/g7, or BDC2.5/NOD-RAG−/− mice are obtained from Bar Harbor, ME, USA or from other sources.

### Method

To confirm the MINC-gadopentetate can be used as a contrast image for pancreatic islets in type 1 diabetes, a mouse model is used. In brief, NOD/Lt, Eα16/NOD, NOD-RAG−/−, BDC2.5/NOD, BDC2.5/B6·H-2g7/g7, or BDC2.5/NOD-RAG−/− mice are bred under specific-pathogen-free conditions. Diabetes monitored by measuring glucose in the urine and then be confirmed by measuring blood glucose levels. The type 1 diabetes mice are i.v. injected with gadopentetate and MINC-gadopentetate at a dose of 0.025 to 250 mmol Gd/kg through, respectively. T2 measurements perform with an 8.5-tesla microimaging system and be reported as R₂ relaxation rates of tissue (R₂₌₁/T2) by standard procedures.

### Example 6. Comparing Contrasting Signal of MINC-Tc99m and Tc99m for SPECT Tumor Imaging (Prophetic Example)

### Objective

This example is to demonstrate that compared to the free Tc99m, MINC-Tc99m improves the contrast signal of tumor regions. SPECT imaging is used to confirm contrast difference between normal and tumor.

### Materials

MINC-Tc99m is Tc99m encapsulated within OEGCG and PEG-EGCG and it is prepared according to WO2006/124000.

Tc99m is obtained from Lantheus medical image, Inc., USA or from other sources.

Male athymic nude mice are obtained from Jackson Laboratories, USA or other sources.

HeLa human ovarian cancer cell line is obtained from ATCC, USA

### Method

To confirm the MINC-Tc99m can be used as a contrast image for tumor detection, an in vivo xenograft tumor model is used. In brief, male 5-week-old nude mice are subcutaneously injected with 1×10⁶ HeLa cells/mouse in the right foreleg. The tumor is expected to have a volume of 0.4-0.7 cm³ at about 3 weeks post-injection. MINC-Tc99m in PBS (0.1 μCi to 500 μCi) then is injected intravenously via the tail vein. The mice are anesthetized by using 2% isoflurane through a mask while on the imaging bed. The tumor-bearing mice are scanned by SPECT at 30, 90, 150, and 240 min post-injection by using a NanoSPECT In Vivo Animal Imager (Bioscan Ltd., Washington, D.C.) with a tube voltage of 80 kV, tube current of 450 ρA, and slice thickness of 45 μm. All image data is reconstructed and analyzed by In Vivo Scope software supplied by the manufacturer.

The encapsulated contrast agent here, Tc99m, can be substituted with I-131, Tc-99m MDP, Tc-99m MAA, Tc-99m PYP, Tc-99m sulfur colloid, I-131 metaiodobenzyguandine (MIBG), T1-201, Ga-67, I-123, Tc-99m 04, TcO4-, Tc-99m phytate, Tc-99m DISIDA, Tc-99m DTPA, Tc-99m MAG3, Tc-99m DMSA, Tc-99m HMPAO, Tc-99m ECD, Tc-99m MIBI, Tc-99m sestamibi, T1-201, 1-131 OIH, I-131 6b-iodomethyl-19-norcholesterol (NP59), Ga-67, Xe-133, Kr-81m, In-111 or I-123 IMP

### Example 7. Comparing Contrasting Signal of MINC-89Zr and 89Zr for PET Tumor Imaging (Prophetic Example)

### Objective

This example is to demonstrate that comparing to the free 89Zr, MINC-89Zr improves the contrast signal of tumor regions. PET imaging is used to confirm contrast difference between normal and tumor.

### Materials

MINC-89Zr is 89Zr encapsulated within OEGCG and PEG-EGCG and it is prepared according to WO2006/124000.

89Zr is obtained from Lantheus medical image, Inc., USA or Cisbio) or from other sources.

Female nude NCr mice are obtained from Jackson Laboratories, USA) or from other sources.

Isoflurane is obtained from Baxter Healthcare, USA or from other sources.

4T1 breast cancer cell line is obtained from ATCC, USA.

### Method

To confirm that MINC-89Zr can be used as a contrast agent for tumor detection, an in vivo xenograft tumor model is used. In brief, female nude NCr mice (8-10 weeks old, n=8) bearing 4T1 breast tumors are injected with 9.3±1.5 MBq (range, 7.8-11.5 MBq) of free 89Zr or MINC-89Zr (3-4 mmol of lipid) in 200-250 mL of PBS solution via the lateral tail vein, respectively. At predetermined time points (2, 24, 48, and 120 h), the animals are anesthetized with a mixture of isoflurane and oxygen gas (2% for induction and 1% for maintenance), and scans then obtained by using an Inveon PET scanner (Siemens Healthcare Global). Whole-body PET static scans recording a minimum of 50 million coincident events are performed, with a duration of 10-20 min. The energy and coincidence timing windows are 350-700 keV and 6 ns, respectively. The image data is normalized to correct for nonuniformity of response of the PET, dead-time count losses, positron branching ratio, and physical decay to the time of injection. The counting rates in the reconstructed images convert to activity concentrations (% ID/g of tissue) by use of a system calibration factor derived from the imaging of a mouse-sized phantom containing 89Zr. Images analyze using ASIPro VM™ software (Concorde Microsystems). Activity concentration quantifies by averaging the maximum values in at least 5 regions of interest drawn on adjacent slices of the tissue of interest.

The encapsulated contrast agent here, 89Zr, can be substituted with rubidium chloride Rb-82, neuraceq, vizamyl, florbetapir F-18, choline C-11, amyvid, Ga-68 dotatate, axumin, flutemetamol F-18, cardiogen-82, florbetaben F-18, fluciclovine F-18, Ruby-Fill, cerianna, netspot, Ga-68 dotatoc, tauvid, Ga-68 psma-11, detectnet, fluoroestradiol F-18, Cu-64 dotatate, flortaucipir F-18, piflufolastat F-18, pylarify, illuccix or locametz.

The invention, and the manner and process of making and using it, are now described in such full, clear, concise, and exact terms as to enable any person skilled in the art to which it pertains, to make and use the same. It is to be understood that the foregoing describes preferred embodiments of the present invention and that modifications may be made therein without departing from the scope of the present invention as set forth in the claims. To particularly point out and distinctly claim the subject matter regarded as the invention, the following claims conclude this specification.

What is claimed is:

1. A diagnostic imaging composition comprising micelles having an outer shell comprising one or more polymer-flavonoid conjugates, optionally an inner shell comprising one or more flavonoid oligomer, and a diagnostic imaging agent encapsulated within the shells;
wherein the polymer is a hydrophilic polymer having a molecular weight of 1,000 to 100,000 daltons, and is selected from the group consisting of: poly(ethylene glycol) (PEG), hyaluronic acid, dextran, polyethylenimine, poloxamers, povidone, D-alpha-tocopheryl, and polyethylene glycol succinate;
the flavonoid is EGCG, EC, EGC, or ECG, as shown in the structures below: 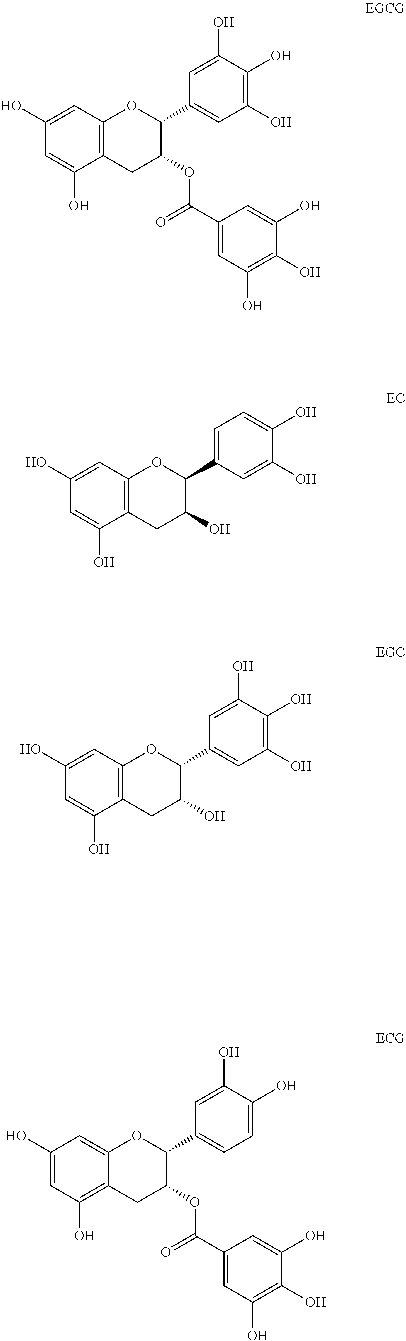 [SMILES: O=C(O[C@@H]1Cc2c(O)cc(O)cc2O[C@@H]1c1cc(O)c(O)c(O)c1)c1cc(O)c(O)c(O)c1 ; Oc1cc(O)c2c(c1)O[C@@H](c1ccc(O)c(O)c1)[C@@H](O)C2 ; Oc1cc(O)c2c(c1)O[C@H](c1cc(O)c(O)c(O)c1)[C@H](O)C2 ; O=C(O[C@@H]1Cc2c(O)cc(O)cc2O[C@@H]1c1ccc(O)c(O)c1)c1cc(O)c(O)c(O)c1]
the flavonoid oligomer comprises 2-20 flavonoids of EGCG, EC, EGC, or ECG; and
the diagnostic imaging agent is a computed tomography (CT) imaging agent, a magnetic resonance imaging (MRI) imaging agent, a positron emission tomography (PET) agent, a single-photon emission computed tomography (SPECT) agent, an ultrasound agent, and a photoacoustic (PA) imaging agent.

2. The diagnostic imaging composition of claim 1, wherein the micelles having an outer shell comprising PEG-EGCG and an inner shell comprising EGCG oligomers.

3. The diagnostic imaging composition of claim 2, wherein the diagnostic imaging agent is a CT imaging agent selected from the group consisting of: iohexol, iodixanol, diatrizoate, metrizoate, iopamidol, iopromide, ioversol, ioxilan, cholografin meglumine, conray, conray 30, conray 43, cysto-conray II, cysto-conray, cystografin dilute, cystografin, gastrografin, renografin-76, ethiodol, hexabrix, or isovue, diatrizoate sodium, meglumin, ioxaglate, ioxaglate, ioxaglate sodium, iothalamate, iron oxide, iothalamate sodium, Bi-DTPA, N1177, Bi-NU-901, PVB-Bi2S3, Er3+-doped Yb203, metrizoate, iobitridol, barium, or and carbon dioxide.

4. The diagnostic imaging composition of claim 2, wherein the diagnostic imaging agent is a MRI imaging agent selected from the group consisting of: gadopentetate, gadoterate, gadodiamide, gadoxetate, gadobutrol, gadoteridol, gadobenate, gadoxetate, gadoversetamide, gadodiamide, gadofosveset, gadopentetic acid dimeglumine, gadoxentate, gadocoletic acid, gadomelitol, gadomer 17, gadoxetic acid, gadoterate meglumine, gadoxetate disodium, gadofosveset trisodium, mangafodipir, gadobenate dimeglumine, ferumoxsil, ferumoxides, iron oxide, EP-3533, ManlCS1, USPIO-g-sLex, MS-325, PVP-IO, ProCA, SPION, SPION-AN-FA, or and Fe3O4.

5. The diagnostic imaging composition of claim 2, wherein the diagnostic imaging agent is a PET imaging agent selected from the group consisting of: 89Zr, Rubidium chloride Rb-82, neuraceq, vizamyl, florbetapir F-18, choline C-11, amyvid, Ga-68 dotatate, axumin, flutemetamol F-18, cardiogen-82, florbetaben F-18, fluciclovine F-18, Ruby-Fill, cerianna, netspot, Ga-68 dotatoc, tauvid, Ga-68 psma-11, detectnet, fluoroestradiol F-18, Cu-64 dotatate, flortaucipir F-18, piflufolastat F-18, pylarify, illuccix, or locametz, C-11, Ga-68, C11-PiB, Cu-64 ATSM, LMI1195, F-18 TFB, F-18 FSPG, F18-FDS, Ga-68-apo-transferrin, F-18 AV-133, F-18 AV-45, F-18-T808, F-18-T807, or and F-18-GE-180.

6. The diagnostic imaging composition of claim 2, wherein the diagnostic imaging agent is a SPECT imaging agent selected from the radioisotopes consisting of: gallium (III), Tc99m, I-131, Tc-99m MDP, Tc-99m MAA, Tc-99m PYP, Tc-99m sulfur colloid, I-131 metaiodobenzyguandine (MIBG), T1-201, Ga-67, I-123, Tc-99m 04, TcO4-, Tc-99m phytate, Tc-99m DISIDA, Tc-99m DTPA, Tc-99m MAG3, Tc-99m DMSA, Tc-99m HMPAO, Tc-99m ECD, Tc-99m MIBI, Tc-99m sestamibi, T1-201, 1-131 OIH, I-131 6b-iodomethyl-19-norcholesterol (NP59), Ga-67, Xe-133, Kr-81m, In-111, or I-123 IMP, Lu-177, I-123 iodoamphetamine, Cu-64, As-72, Zr-89, I-124, C-11, Ga-68, F-18, Tc-99m CN5DG, Ro 16-0154, In111-DOTA-5D3, Tc99m-HYNIC-TMTP1 or QT-DTC-bisbiotin.

7. The diagnostic imaging composition of claim 6, wherein the radioisotope is attached to a ligand.

8. A method for diagnostic imaging, comprising:
administering to a subject in need thereof an effective amount of the diagnostic imaging composition of claim 3;
performing computed tomography (CT) scan,
measuring the difference in attenuating x-rays between lesions and normal tissues, and
determining the location and/or size of lesions.

9. The method of claim 8, wherein the CT scan is performed in whole body, brain, head, chest, neck, spine, sinus, pelvic, or abdomen.

10. The method of claim 8, wherein the lesions are tumors, autoimmune diseases, cardiovascular diseases, central nervous diseases, infection, and inflammatory lesions.

11. The method of claim 8, wherein the diagnostic imaging agent is administered intravenously.

12. A method for diagnostic imaging, comprising:
administering to a subject in need thereof an effective amount of the diagnostic imaging composition of claim 4,
performing magnetic resonance imaging (MRI),
measuring the differences in magnetic resonance signals generated by magnetic fields between lesions and normal tissues to detect lesions; and
determining the location and/or size of diseased lesions.

13. The method of claim 12, wherein the lesions are tumors, autoimmune diseases, cardiovascular diseases, central nervous diseases, infection, and inflammatory lesions.

14. The method of claim 12, wherein the diagnostic imaging agent is administered intravenously.

15. A method for diagnostic imaging, comprising:
administering to a subject in need thereof an effective amount of the diagnostic imaging composition of claim 5,
performing positron emission tomography (PET) imaging,
measuring three-dimensional image formed by the gamma rays resulted from positron emitted by the imaging agent, and
determining the location and/or size of lesions.

16. The method of claim 15, wherein the lesions are tumors, autoimmune diseases, cardiovascular diseases, central nervous diseases, infection, and inflammatory lesions.

17. The method of claim 15, wherein the diagnostic imaging agent is administered intravenously.

18. A method for diagnostic imaging, comprising:
administering to a subject in need thereof an effective amount of the diagnostic imaging composition of claim 6,
performing single-photon emission computed tomography (SPECT) imaging,
measuring the differences in signal intensity of gamma rays between lesions and normal tissues, and
determining the location and/or size of lesions.

19. The method of claim 18, wherein the lesions are tumors, autoimmune diseases, cardiovascular diseases, central nervous diseases, infection, and inflammatory lesions.

20. The method of claim 18, wherein the diagnostic imaging agent is administered intravenously.
